(19) United States
(12) Patent Application Publication
(10) Pub. No.: US 2015/0050227 A1
(43) Pub. Date: Feb. 19, 2015

(54) Compositions Having Perspiration Reduction Properties

(71) Applicant: AMCOL INTERNATIONAL CORPORATION, Hoffman Estates (IL)

(72) Inventors: Limin Liu, Palatine, IL (US); Ralph Spindler, Palatine, IL (US); Thomas W. Beihoffer, Arlington Heights, IL (US); Trining Renfro, Carol Stream, IL (US)

(21) Appl. No.: 14/382,365

(22) PCT Filed: Mar. 4, 2013
(86) PCT No.: PCT/US13/28952

§ 371 Date: Sep. 2, 2014

Related U.S. Application Data
(60) Provisional application No. 61/606,165, filed on Mar. 2, 2012.

(51) Int. Cl. A61K 8/26; A61K 8/28; A61Q 15/00; A61K 8/02; A61K 8/81

(52) U.S. Cl. CPC A61K 8/26; A61K 8/0241; A61K 8/8152; A61Q 15/00; A61K 8/28; A61K 2800/60
USPC 424/66; 424/68

(57) ABSTRACT

A composition containing polymeric microparticles is disclosed. The composition effectively reduces perspiration in the absence of aluminum/zirconium antiperspirant compounds.

### CROSS-REFERENCE TO RELATED APPLICATION

The benefit under 35 U.S.C. §119(e) of U.S. Provisional Patent Application No. 61/606,165 filed Mar. 2, 2012, is hereby claimed and the disclosure is incorporated herein by reference in its entirety.

### BACKGROUND

1. Field of the Disclosure

The present disclosure is directed to compositions containing polymeric microparticles that effectively reduce perspiration to control underarm wetness and malodor formation. The compositions optionally can further contain an antiperspirant compound, like an astringent salt, and/or additional deodorants. The present invention also is directed to methods of using the deodorant compositions.

2. Brief Description of Related Technology

Perspiring assists in the regulation of body temperature and moisturizes skin. Perspiration also leads to the generation of undesired malodors. The malodors that develop from human perspiration primarily are the result of microbial interactions with sweat gland secretions, which produce odor-causing small chain fatty acids.

Deodorants are products that reduce axillary malodor, whereas antiperspirants are used to reduce perspiration. Deodorant and antiperspirant compositions are well known in the personal care arts to control body malodors associated with human perspiration. Deodorant compositions can contain deodorant actives, such as antimicrobial agents, to help control the generation of malodors and/or can contain fragrances that help mask the sensory perception of malodors. Antiperspirants are effective, but do not completely eliminate perspiration. Therefore, body odor can result even when an antiperspirant or deodorizing composition is used on a continuous basis.

Deodorant and antiperspirant compositions are available in a variety of forms, such as aerosol suspensions, pump sprays, roll-on, solutions, dispensers, liquid emulsions, solid gels, waxes, and suspensions. Deodorant and antiperspirant compositions are used by rubbing or spraying an area of the body, such as the underarm, to apply a layer of the composition to the skin, and thereby reduce odor, and in some cases, perspiration. An ideal deodorant or antiperspirant composition is stable for the life of the composition, effectively delivers compounds to the skin, and is esthetically pleasing to the consumer.

A number of antiperspirant actives are available, including aluminum salts, zirconium salts, and aluminum/zirconium salts, to reduce sweat production. However, topical use of aluminum, zirconium, and aluminum/zirconium salts poses safety concerns, as disclosed in publications relating adverse effects arising from the use of antiperspirants. Further, the acidity of aluminum, zirconium, and aluminum/zirconium salts makes them potential skin irritants. The aluminum, zirconium, and aluminum/zirconium antiperspirants also are sensitive to metals, such as iron, which can destabilize the antiperspirant and cause color formation and fabric stains.

Over the past decade, the deodorant/antiperspirant market has shown a mild, steady growth. Although the search for a technological breakthrough in the art continues, the focus of research and development is primarily directed to improving existing product lines and using different marketing approaches, such as gender and age segmentation. Some recent deodorant/antiperspirant products incorporate provitamins to improve skin smoothness, or prolong malodor control by using a sustained deodorizing delivery system. One recent advance in the art is use of philodendron plant extract to inhibit bacterial degradation of perspiration.

Deodorant/antiperspirant compositions often are formulated as sticks that also contain a gellant or other structurant, and a polar alcohol to help solubilize the gellant or other structurant. Such compositions usually are topically applied to the underarm or other area of the skin. Although these deodorant/antiperspirant sticks are quite popular and commonly used to control perspiration or mask malodors associated with human perspiration, these alcohol-containing sticks can cause skin irritation, burning, and itching after topical application. The irritation, burning, and itching caused by such stick products make them less suitable for application to other, more sensitive, areas of the body.

Other attempts at controlling body malodors include the use of odor absorbers, such as activated charcoals and zeolites. Cyclodextrins also have been used in deodorant compositions for topical application. Deodorant compositions that contain these malodor absorbing agents often are formulated as aqueous lotions, aqueous roll-ons, and aqueous soft deodorant gels, which comprise the odor absorber and an aqueous liquid carrier. However, activated charcoal and zeolite odor absorbing agents may be ineffective when wet and are known to be less efficient at absorbing odors when included in aqueous systems, especially when the aqueous compositions are applied to the skin and the activated charcoal or zeolite comes in contact with human body fluids, such as sweat.

Numerous patents disclose the use of polymeric absorbents to control perspiration, for example, U.S. Pat. Nos. 5,408,705; 4,863,721; and 4,659,564; and U.S. Patent Publication Nos. 2008/0247978 and 2009/0087396. However, the absorbents disclosed in this patents and publications swell to form gels when contacted with water, and therefore cannot be readily formulated into aqueous compositions. The compositions also may leave a “tacky” feel on the skin.

U.S. Pat. Nos. 5,281,413, and 5,387,411 disclose the use of adsorbent polymeric microparticles in anhydrous antiperspirant compositions further containing a high amount of antiperspirant actives, such as aluminum/zirconium salts.

A need therefore still exists in the art for compositions that reduce the generation of perspiration and the malodors associated with perspiration, especially aqueous compositions and compositions containing no or low amounts of an antiperspirant active. The present invention is directed to compositions and methods that reduce perspiration, and thereby control or eliminate malodors resulting from perspiration.

### SUMMARY

The present invention relates to compositions having sufficient efficacy in reducing perspiration to control perspiration-related malodors, and to methods of using the compositions. More particularly, the present invention is directed to compositions comprising polymeric microparticles. A present composition effectively reduces perspiration, and thereby perspiration-related wetness and malodors, either in the absence of an aluminum-based antiperspirant active or in combination with low amounts of an aluminum-based antiperspirant active.

The present disclosure also relates to methods of reducing perspiration and controlling the wetness and malodors associated with human perspiration, especially underarm odor. The methods comprise topically applying an effective amount of a composition of the present invention to the skin of a human.

In one embodiment of the disclosure, the composition is free or substantially free of an aluminum-based antiperspirant active. In another embodiment of the invention, the composition optionally contains a low amount of an aluminum-based antiperspirant active. In this embodiment, the presence of the polymeric microparticles allows a decrease in the amount of the aluminum-based antiperspirant active in the composition to achieve the same degree of perspiration reduction as a greater amount of an aluminum-based antiperspirant active in the absence of the polymeric microparticles. As a result, a present composition eliminates, or at least abates, the adverse effects of an aluminum-based antiperspirant active.

An aspect of the present disclosure therefore is to provide a composition comprising polymeric microparticles. In some embodiments, the composition further comprises a suspending agent for the microparticles. Another aspect of the present invention is to provide a composition comprising polymeric microparticles, wherein the microparticles are free of an entrapped, or loaded, aluminum-based antiperspirant active. In some embodiments, the composition as a whole is free of an aluminum-based antiperspirant active.

Still another aspect of the present disclosure is to provide a composition that imparts an effective perspiration reduction for at least 24 hours after application of the composition to the skin. Another aspect of the present invention is to provide a composition that exhibits a mean sweat reduction of at least 20% one hour after application and/or at least 20% 24 hours after application, in the absence of an aluminum-based antiperspirant active.

Yet another aspect of the present disclosure is to provide a composition in the form of a solid stick, roll-on, a gel, or a lotion. In another aspect, the present disclosure is directed to a wet wipe comprising one or more layers of a substrate and a composition of the present disclosure. Still another agent is to provide aqueous, non-aqueous, and emulsified compositions useful as antiperspirants.

In accordance with an embodiment of the disclosure, a sweat reducing composition includes an aluminum-based antiperspirant active in an amount of less than 15% by weight based on the total weight of the composition, about 1% to about 20%, by weight, of water and oil adsorbing polymeric microparticles, and a carrier.

In accordance with another embodiment of the disclosure, a sweat reducing composition consists of water and oil adsorbing polymeric microparticles in an effective amount to reduce the generation of human perspiration in at least 50% of users tested by at least 10% one hour after contacting the skin and/or by at least 10% twenty-four hours after contacting the skin, a carrier, and optionally one or more of an aluminum-based antiperspirant active in amount less than 15% by weight based on the weight of the composition, an odor control active material, an emollient, a fragrance, a dye, a moisturizer, a skin protectant, an antimicrobial, a cosmetic biocide, an opacifier, a surfactant, a gelling or thickening agent, a suspending agent, and a film-forming polymer. The reduction in perspiration is measured in accordance with the testing protocol defined at 21 C.F.R. §350.60.

In accordance with yet another embodiment, a wet wipe can include a water-insoluble substrate; and a composition of any one of the preceding paragraphs.

In accordance with an embodiment of the disclosure, a method of reducing the generation of human perspiration includes contacting human skin with a composition in an effective amount to reduce the generation of human perspiration. The composition includes less than 15% by weight of an aluminum-based antiperspirant active, about 1% to about 20%, by weight, of water and oil adsorbing polymeric microparticles, and a carrier.

In accordance with another embodiment of the disclosure, a method of reducing the generation of human perspiration includes contacting human skin with a composition comprising about 1% to about 20%, by weight, of water and oil adsorbing polymeric microparticles and a carrier, in an effective amount to reduce the generation of human perspiration, wherein the composition provides a reduction in the perspiration that is greater than the adsorbent capacity of the polymeric microparticles.

These and other features, aspects, and advantages of the present disclosure will become evident to those skilled in the art from the following detailed description of the preferred embodiments.

### BRIEF DESCRIPTION OF THE DRAWINGS

FIG. 1 is a graph illustrating the results of user comparative testing of a composition in accordance with the disclosure and a conventional product.

### DETAILED DESCRIPTION

A present composition comprises polymeric microparticles dispersed in carrier. The carrier can be aqueous, non-aqueous, or aqueous and non-aqueous in the form of an emulsion. The composition can contain additional ingredients typically present in a deodorant/antiperspirant composition. A present composition can be free or substantially of an aluminum-based antiperspirant active or optionally can contain an aluminum-based antiperspirant active, for example, in a reduced amount as compared to conventional antiperspirant compositions. The compositions effectively reduce perspiration, and therefore the feeling of wetness and the malodors associated with perspiration. The compositions of the disclosure can be applied to reduce human perspiration. Methods of using the composition of the disclosure include applying the composition to human skin, for example, by contacting skin with the composition, in an amount effective to reduce the generation of human perspiration.

In embodiments containing an aluminum based antiperspirant active, the amount of the aluminum based antiperspirant active in the composition can be reduced, i.e., less than 15 wt %, less than 14 wt %, less than 13 wt %, less than 12 wt %, less than 11 wt %, less than 10 wt %, less than 9 wt %, less than 8 wt %, less than 7 wt %, less than 6 wt %, less than 5 wt %, less than 4 wt %, less than 3 wt %, less than 2 wt %, less than 1 wt %, based on the total weight of the composition, for example, due to the presence of the polymeric microparticles. In embodiments, the compositions that are substantially free or contain a reduced amount of an aluminum based antiperspirant active achieves the same efficacy as a composition that is (a) free of polymeric microparticles and (b) contains a greater amount of the aluminum-based antiperspirant active, for example, greater than 15wt % or greater than 20 wt % of an aluminum-based antiperspirant active, typically used in conventional antiperspirant compositions.

As used herein, “aluminum-based antiperspirant active” means an aluminum containing compound that has demonstrated an ability to reduce sweat production, for example, but not limited to, astringent salts like aluminum and aluminum/zirconium salts. As described in 21 C.F.R. §350.10 (2003), the disclosure of which is incorporated herein by reference in its entirety, conventional aluminum-based antiperspirant actives include, aluminum chloride, aluminum chlorohydrate, aluminum chlorohydrex propylene glycol, aluminum dichlorohydrate, aluminum dicholorohydraex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrex, aluminum zirconium octachlorohydrex gly, aluminum zirconium pentachlorohydrate, aluminum zirconium pentachlorohydrex gly, aluminum zirconium tetrachlorohydrate, aluminum zirconium tetrachlorohydrex gly, aluminum zirconium trichlorohydrate, aluminum zirconium tricholorhydrex gly, and combinations thereof.

A present composition reduces the generation of perspiration, and accordingly provides a wetness and body malodor control without skin irritation and provides a long lasting sweat reduction benefit after the composition has been topically applied to the skin. The terms “body odor” and “body malodor”, as used herein, mean odors generated as a result of the natural functioning of a human or mammalian body. Such odors include, but are not limited to, odors produced by microorganisms on the human or mammalian skin (i.e., bacterial decomposition of skin secretions). Body odors are primarily attributed to organic compounds that have various structures and functional groups, such as amines, acids, alcohols, aldehydes, ketones, phenolics, and polycyclics including aromatics and polyaromatics. The odor-causing compounds also can include sulfur-containing functional groups, such as thiol, sulfide, and/or disulfide groups.

The term “sweat reduction” and “antiperspirant” mean an activity of reducing the generation of perspiration as determined by FDA Guidelines §350.60, 21 CFR Parts 310, 350, and 369 (2003).

In accordance with embodiments of the disclosure, the composition can cause an at least 10%, 15%, 20%, 25%, or 30% reduction in human perspiration one hour after contacting the skin, in at least 50% of users tested, as measured by the FDA protocol provided at 21 C.F.R. §350.60. In accordance with embodiments of the disclosure, the composition can also or alternatively cause an at least 10%, 15%, 20%, 25%, or 30% reduction in human perspiration twenty-four hour after contacting the skin, in at least 50% of users tested, as measured by the FDA protocol provided at 21 C.F.R. §350.60.

The term “sweat reduction” and “antiperspirant” mean an activity of reducing the generation of perspiration as determined by the testing protocol defined in the FDA Guidelines at, 21 C.F.R. §350.60 (2003), the disclosure of which is incorporated herein by reference in its entirety. Unless otherwise stated, the percentage of sweat reductions reported through the disclosure are sweat reduction percentages as measured by the FDA Guidelines, which refers to at least 50% of the users tested demonstrating the asserted sweat reduction percentage.

Without intending to be bound by theory, it is believed that a composition of the disclosure reduces the generation of perspiration, in part, by adsorbing perspiration prior to bacterial action that degrades components of perspiration resulting in malodorous products. It is further theorized that the adsorbed perspiration may be shielded from bacterial action. It is further theorized that the polymeric microparticles form a film on the skin to reduce the amount of perspiration.

As an additional benefit, because antiperspirant actives, such as aluminum or aluminum/zirconium salts, are not required in a present composition, the potential for skin irritation is greatly reduced. Effective sweat-reducing compositions free of an astringent salt provide benefits, such as reducing or eliminating contact dermatitis, and eliminating contact with aluminum salts that are connected with breast cancer, Alzheimer's disease, and clogged sweat pores leading to infected sweat glands. The polymeric microparticles also reduce greasiness of the composition to provide a dryer, powdery feel during use of application, thereby improving composition esthetics. In addition, by eliminating a source of perspiration-related malodors, the present compositions outperform deodorants that merely mask malodors.

Without intending to be bound by theory, it is believed that the microparticles contained in a present composition reduce the generation of perspiration, in part, by adsorbing a portion of the perspiration, and it is further believed that the high surface area of microparticles may help facilitate and/or expedite the evaporation of perspiration to achieve the reduction of perspiration beyond the adsorption capacity of the microparticles themselves.

In various embodiments, the composition of the disclosure provide a reduction in the amount of human perspiration that is greater than the adsorbent capacity of the polymeric microparticles. For example, in various embodiments, the composition provides an at least 20% reduction in the perspiration in 24 hours for at least 50% of the users tested, and the microparticles adsorb less than 25% of the perspiration based on the amount perspiration reduced in order to provide at least 20% reduction as measured in accordance with the testing protocol defined at 21 C.F.R. §350.60. In accordance with another embodiment, the microparticles of the composition adsorb less than 25% of the perspiration based on the amount of perspiration reduced by the composition in a 24 hour period.

As used herein, the terms “a hydrophilic compound” and “a hydrophilic agent” mean the compound or agent has a water solubility of at least 0.1 grams per 100 grams of water at 25° C. The terms “a hydrophobic formulation” and “a hydrophobic agent” means the compound or agent has a water solubility of less than 0.1 grams per 100 grams of water at 25° C.

A present composition comprises about 1% to about 20%, by weight of the composition, of polymeric microparticles. In a preferred embodiment, the composition comprises about 2% to about 18%, by weight of the composition, of polymeric microparticles. In another preferred embodiment, the composition comprises about 3% to about 18%, by weight of the composition, of polymeric microparticles. In various embodiments, a present composition contains 1%, 2%, 3%, 4%, 5%, 6%, 7%, 8%, 9%, 10%, 11%, 12%, 13%, 14%, 15%, 16%, 17%, 18%, 19%, or 20%, and all ranges and subranges therein, by weight of the composition, of polymeric microparticles.

One class of adsorbent polymeric microparticles useful in a present composition is prepared by a suspension polymerization technique, as set forth in U.S. Pat. Nos. 5,677,407; 5,712,358; 5,777,054; 5,830,967; and 5,834,577, each incorporated herein by reference. Such an adsorbent polymer is sold under the tradename of POLY-PORE® E200 (INCI name: allyl methacrylate crosspolymer) available from AMCOL International Corporation, Arlington Heights, Ill.

A further useful class of adsorbent microparticle polymers is prepared by a precipitation polymerization technique, as set forth in U.S. Pat. Nos. 5,830,960; 5,837,790; 6,248,849; and 6,387,995, each incorporated herein by reference. Such an adsorbent polymer is sold under the tradename POLYTRAP® 7603 (INCI name: glycol dimethacrylate/alkyl methacrylate crosspolymer), also available from AMCOL International Corp.

A further useful class of adsorbent polymers prepared by a precipitation polymerization technique is disclosed in U.S. Pat. Nos. 4,962,170; 4,948,818; and 4,962,133, each incorporated herein by reference, and are commercially available under the tradename of POLYTRAP® 6603 (INCI name: lauryl methacrylate/glycol dimethacrylate crosspolymer), also available from AMCOL International Corp. Other useful, commercially available adsorbent polymers include, for example, MICROSPONGE® (INCI name: methyl methacrylate/glycol dimethacrylate crosspolymer), disclosed in U.S. Pat. No. 4,690,825 and available from AMCOL International Corp., and Poly-HIPE polymers (e.g., a copolymer of 2-ethylhexyl acrylate, styrene, and divinylbenzene) available from Biopore Corporation, Mountain View, Calif.

In particular, adsorbent polymeric microparticles prepared by the suspension polymerization technique, e.g., POLY-PORE® E200, are a highly porous and highly crosslinked polymer in the form of open (i.e., broken) spheres and sphere sections characterized by a mean unit particle size of about 0.5 to about 3,000 microns, preferably about 0.5 to about 300 microns, more preferably about 0.5 to about 100 microns, and most preferably about 0.5 to about 80 microns. A significant portion of the spheres is about 20 microns in diameter.

The polymeric microparticles are oil and water adsorbent, and have an extremely low bulk density of about 0.008 gm/cc to about 0.1 gm/cc, preferably about 0.009 gm/cc to about 0.07 gm/cc, and more preferably about 0.0095 gm/cc to about 0.04-0.05 gm/cc. The microparticles are capable of adsorbing hydrophobic compounds, as well as hydrophilic compounds, individually, or both hydrophobic and hydrophilic compounds simultaneously.

The adsorbent polymer microparticles prepared by the suspension polymerization technique include at least two polyunsaturated monomers, preferably allyl methacrylate and an ethylene glycol dimethacrylate, and, optionally, monounsaturated monomers in an amount of 0 to 10 mol % of the total monomer content. The microparticles are characterized by being open to their interior, due either to particle fracture upon removal of a porogen after polymerization or to subsequent milling. The microparticles have a mean unit diameter of less than about 50 microns, preferably less than about 25 microns, and have a total adsorption capacity for organic liquids, e.g., mineral oil, that is at least about 72% by weight, preferably at least about 93% by weight, and an adsorption capacity for hydrophilic compounds and aqueous solutions of about 70% to about 89% by weight, preferably about 75% to about 89% by weight, calculated as weight of material adsorbed divided by total weight of material adsorbed plus dry weight of polymer. In a preferred embodiment, the broken sphere microparticles are characterized by a mean unit diameter of about 1 to about 50 microns, more preferably of about 1 to about 25 microns, most preferably, of about 1 to about 20 microns.

Preferred polymeric microparticles comprise a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, a copolymer of methyl methacrylate and glycol methacrylate, and mixtures thereof.

Specific polymeric microparticles useful in the present invention include the previously described POLY-PORE® E200, POLYTRAP® 7603, POLYTRAP® 6603, MICROSPONGE®, and Poly-HIPE particles, for example.

The polymeric microparticles are used “as is” in the present composition to achieve effective sweat reduction, i.e., are free of an intentionally entrapped or loaded aluminum-based antiperspirant active.

In another embodiment, the polymeric microparticles are loaded with one or more cosmetic or therapeutic compound prior to being formulated into a present composition. The loaded or entrapped microparticles still effectively achieve an effective sweat reduction due to the large capacity of the polymeric microparticles to adsorb large amounts of different materials without an adverse effect on the antiperspirant capabilities of the polymeric microparticles. A cosmetic or therapeutic compound therefore is loaded onto the polymeric microparticles in a sufficient amount to provide microparticles containing about 10% to about 90%, preferably about 20% to about 80%, by weight, of the cosmetic or therapeutic compound.

In accordance with embodiments the present disclosure, an aluminum-based antiperspirant active is not incorporated into the polymeric particles. The cosmetic or therapeutic compounds incorporated into the polymeric particles include, but are not limited to, the ingredients disclosed below as optional ingredients present in a deodorant composition of the invention.

A present composition is prepared by merely admixing the polymeric microparticles with the other composition ingredients, with heating if necessary. The other composition ingredients include carriers, such as volatile and/or non-volatile organic solvents or water and water-soluble solvents, suspending agents, such as polymeric elastomers, dyes, fragrances, emulsifiers, and other ingredients typically included deodorant compositions. Various composition ingredients are discussed below in more detail.

In embodiments wherein a cosmetic or therapeutic compound is entrapped or loaded into the polymeric microparticles, the loaded microparticles first are prepared, then the loaded microparticles are formulated to provide a present composition.

As used herein, the term “loaded microparticle” refers to a microparticle having a cosmetic or therapeutic compound added thereto. Loading of the cosmetic or therapeutic compound includes one or more of impregnating, imbedding, entrapping, absorbing, and adsorbing of the cosmetic or therapeutic compound into or onto the polymeric microparticles. Loading of the cosmetic or therapeutic compound also can be referred to as an “entrapment.” The term entrapment refers to a physical loading of the cosmetic or therapeutic compound onto the microparticles. Loading can be accomplished by spraying or adding the cosmetic or therapeutic compound directly to the microparticles in a manner such that a homogeneous distribution of the cosmetic or therapeutic compound on the microparticles is achieved.

Alternatively, the cosmetic or therapeutic compound first can be dissolved in a suitable solvent, then the resulting solution is sprayed or added to the microparticles. The solvent then is removed by heating, vacuum, or both. Two or more different types of materials can be added to the microparticles, wherein one of the cosmetic or therapeutic compounds is an active agent and the other compound is used to modify the release rate of the active agent from the microparticles and/or to protect the active agent loaded in the microparticles from reacting or otherwise interacting with other ingredients contained in the final composition. These release modifying or protective materials can be added in a liquid or molten state directly to the microparticles, or first dissolved in a suitable solvent, sprayed onto the microparticles, followed by removal of the solvent from the delivery system. Alternatively, an active agent can be mixed with a release modifying or protective material, then the resulting mixture is loaded on a microparticle delivery system.

After loading a cosmetic or therapeutic compound on the microparticles, a barrier layer, optionally, can be applied to the loaded microparticles to prevent rapid diffusion of the cosmetic or therapeutic compound from the microparticles, and to protect the cosmetic or therapeutic compound from the surrounding environment until application to the skin. The barrier material has an identity and is present in an amount such that the ability of the polymeric microparticles to reduce perspiration is not adversely affected.

Examples of materials that can be used as a barrier layer, also termed a secondary loading or secondary entrapment, include, but are not limited to, low melting alcohols (C₈ through C₂₀) and fatty alcohols ethoxylated with one to three moles of ethylene oxide. Examples of fatty alcohols and alkoxylated fatty alcohols include, but are not limited to, behenyl alcohol, caprylic alcohol, cetyl alcohol, cetaryl alcohol, decyl alcohol, lauryl alcohol, isocetyl alcohol, myristyl alcohol, oleyl alcohol, stearyl alcohol, tallow alcohol, steareth-2, ceteth-1, cetearth-3, and laureth-2. Additional fatty alcohols and alkoxylated alcohols are listed in the International Cosmetic Ingredient Dictionary and Handbook, Tenth Edition, Volume 3, pages 2127 and pages 2067-2073 (2004), incorporated herein by reference.

Another class of materials that can be used a barrier layer is the C₈ to C₁₂ fatty acids, including, but not limited to, stearic acid, capric acid, behenic acid, caprylic acid, lauric acid, myristic acid, tallow acid, oleic acid, palmitic acid, isostearic acid and additional fatty acids listed in the International Cosmetic Ingredient Dictionary and Handbook, Tenth Edition, Volume 3, page 2126-2127 (2004), incorporated herein by reference. The barrier material also can be a hydrocarbon, like mineral oil, 1-decene dimer, polydecene, paraffin, petrolatum, vegetable-derived petrolatum or isoparafin. Another class of barrier materials is waxes, like mink wax, carnauba wax, and candelilla wax, for example, and synthetic waxes, like silicone waxes, polyethylene, and polypropylene, for example.

Fats and oils can be used as a barrier material, including, but are not limited to, lanolin oil, linseed oil, coconut oil, olive oil, menhaden oil, castor oil, soybean oil, tall oil, rapeseed oil, palm oil, and neatsfoot oil, and additional fats and oils listed in the International Cosmetic Ingredient Dictionary and Handbook, Tenth Edition, Volume 3, pages 2124-2126 (2004). Other useful classes of barrier materials include a water-insoluble ester having at least 10 carbon atoms, and preferable 10 to about 32 carbon atoms. Numerous esters are listed in International Cosmetic Ingredient Dictionary and Handbook, Tenth Edition, Volume 3, pages 2115-2123 (2004).

The barrier layer, if present at all, can be about 1 to about 60 wt. %, by total weight of the loaded polymeric microparticles. In a preferred embodiment, the barrier layer is present at about 5 to about 50 wt. %, and more preferably about 10 to about 40 wt. %, by total weight of the loaded polymeric microparticles.

A composition of the present invention therefore comprises polymeric microparticles optionally loaded with a cosmetic or therapeutic compound and an optional barrier material. A wide variety of cosmetic or therapeutic compounds can be loaded onto polymeric microparticles, and thereby included in a composition of the present invention. More particularly, a cosmetic and therapeutic compound loaded onto the polymeric microparticles can be one of, or a mixture of, a cosmetic compound, a medicinally active compound, or any other compound that is useful upon topical application to the skin. Such cosmetic and therapeutic compounds include, but are not limited to, deodorants, skin-care compounds, antioxidants, antibacterial compounds, antifungal compounds, anti-inflammatory compounds, topical anesthetics, and other cosmetic and therapeutic topically effective compounds.

Topically active drugs, like antifungal compounds, antibacterial compounds, anti-inflammatory compounds, topical anesthetics, skin rash, skin disease, and dermatitis medications, and anti-itch and irritation-reducing compounds can be loaded onto the polymeric microparticles. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidoneiodine, polymyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; anti-inflammatory agents, such as alclometasone dipropionate, beta-methasone valerate, and the like; burn relief ointments, such as o-amino-p-toluene-sulfonamide monoacetate, and the like; depigmenting agents, such as monobenzone; dermatitis relief agents, such as the active steroid amcinonide, diflorasone diactate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration and suitable for use in a deodorant composition, also can be incorporated in a composition of the present invention in an amount sufficient to perform its intended function. Other topically active compounds are listed in Remington's Pharmaceutical Sciences, 17th Ed., Merck Publishing Co., Easton, Pa. (1985), pages 773-791 and pages 1054-1058 (hereinafter Remington's), incorporated herein by reference.

In addition to the polymeric microparticles, either “as is” or loaded with a cosmetic or therapeutic compound, the present compositions further comprise a carrier, which can be aqueous and/or non-aqueous, and other ingredients commonly used deodorant/antiperspirant compositions, such as surfactants, dyes, formulation aids, and solvents. Such additional ingredients are admixed with the polymeric microparticles subsequent to any loading of a cosmetic or therapeutic compound onto the polymeric particles. During the preparation of a present composition, a portion of various additional ingredients may be absorbed or adsorbed by the polymeric particles. However, the majority of the additional formulation ingredients remain in the final composition in the “free form”, i.e., not loaded onto the polymeric microparticles, to perform their intended function without adversely affecting the ability of the polymeric microparticles to reduce perspiration.

The carrier of the present composition can be aqueous or non-aqueous. In some embodiments, the composition is an emulsion containing both aqueous and non-aqueous components. The emulsion can be an oil-in-water (o/w) emulsion or a water-in-oil emulsion (w/o).

In one embodiment, a present composition is free, or essentially free, of water. The carrier therefore comprises water-insoluble, oil-soluble, hydrophobic compounds. Such water-insoluble compounds are present as a carrier of the composition, but also can provide a specific benefit, such as faster drying time, better skin feel, or ease of application.

The carrier can be, for example, an aliphatic hydrocarbon, a water-insoluble ester, a water-insoluble ether, a fatty (C₈-C₁₂) alcohol, a siloxane, or mixtures thereof. These hydrophobic compounds improve the feel of the composition on the skin, allow easier application of the composition to the skin, and allow the skin to dry faster after application of the composition.

Hydrophobic aliphatic hydrocarbons include, for example, hydrogenated polybutenes, isoeicosane, isohexadecene, 1-decene dimer, mineral oils, nonvolatile hydrocarbon fluids, and hydrocarbons depicted in general structural formula (I), wherein n ranges from 2 to 5,

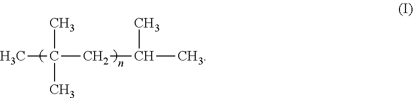

Volatile hydrocarbons, such as a hydrocarbon including about 10 to about 30 carbon atoms, have sufficient volatility to slowly volatilize from the skin after application of the composition. The volatile hydrocarbons provide benefits such as lubrication, a rich feel during application, and faster drying. Specific volatile hydrocarbons having the structural formula (I) are the commercially-available compounds PERMETHYL 99A and PERMETHYL 101A, corresponding to compounds of general structure (I) wherein n is 2 and 3, respectively, and PERMETHYL 102A, available from Permethyl Corporation, Pottstown, Pa.

Siloxanes carriers included in the compositions provide the same benefits as the aliphatic hydrocarbons. Exemplary siloxanes include phenyltrimethicone; cyclic or linear, low molecular weight, volatile polydimethylsiloxanes known as cyclomethicones and dimethicones, respectively; and methicones. The cyclomethicones are low viscosity, low molecular weight, water-insoluble cyclic compounds having an average of about 3 to about 6 —[O—Si(CH₃)₂]— repeating group units per molecule. Cyclomethicones are available commercially under the tradenames SILICONE 344 FLUID and SILICONE 345 FLUID from Dow Corning Corporation, Midland, Mich., and SILICONE SF-1173 and SILICONE 8F-1202 from General Electric, Waterford, N.Y., for example.

An example of a linear, low molecular weight, volatile dimethicone is the compound hexamethyldisiloxane, available commercially under the tradename DOW CORNING 200 FLUID, from Dow Corning Corp., Midland, Mich. DOW CORNING 200 FLUID has a viscosity of 0.65 cs (centistokes), is highly volatile, is nongreasy, and provides lubrication for topical application of the composition of the present invention to the skin. Other linear polydimethylsiloxanes, such as decamethyltetrasiloxane, octamethyltrisiloxane, and dodecamethylpentasiloxane, also have sufficient volatility to provide a dry feel after application. Other useful linear siloxanes are hexyl dimethicone, polyphenylmethylsiloxane, and bisphenylhexamethicone. Nonvolatile siloxanes also can be used as the carrier. The volatile siloxanes and aliphatic hydrocarbons can be used alone, in combination, or in combination with nonvolatile siloxanes and/or nonvolatile aliphatic hydrocarbons.

Other suitable hydrophobic compounds include waxes, oils, and fats, and water-insoluble emollients, like fatty (C₈-C₂₂) alcohols, ethers, and esters. Exemplary hydrophobic compounds include, but are not limited to, dioctyl adipate, isopropyl myristate, isopropyl palmitate, isostearyl benzoate, and polypropylene-15 stearyl ether. Typical emollients are listed in the CTFA Handbook at pages 23 through 28, incorporated herein by reference.

A carrier of a present composition also can be water. In such embodiments, a water-soluble solvent also can be present as a component of the carrier. Therefore, a present composition also can contain an organic solvent. The solvent can be an organic compound containing one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, propylene carbonate, dimethyl isosorbide, dipropylene glycol, tripropylene glycol, hexylene glycol, butylene glycol, pentylene glycol, propylene carbonate, dimethyl isosorbide, 1,2,6-hexanetriol, sorbitol, PEG-4, similar hydroxy-containing compounds, and mixtures thereof. The solvent also can be an aprotic solvent, e.g., dimethyl sulfoxide or tetrahydrofuran.

In other embodiments, the carrier can be a blend of aqueous and non-aqueous materials wherein the composition is in the form of an emulsion containing hydrophobic and hydrophilic solvents, either as an o/w emulsion or a w/o emulsion. The emulsified compositions further comprise an emulsifying agent, for example, a surfactant.

An optional ingredient for inclusion in a present composition can be an aluminum-based antiperspirant active. An aluminum-based antiperspirant active is included in the composition in an amount is less than an amount of the aluminum-based antiperspirant active typically included in a conventional composition in the absence of polymeric microparticles. An antiperspirant active is included in a present composition, if at all, in an amount of 0% to less than 15%, less than 14%, less than 13%, less than 12%, less than 11%, less than 10%, less than 9%, less than 8%, less than 7%, less than 6%, less than 5%, less than 4%, less than 3%, less than 2%, or less than 1%, by weight of the composition.

The aluminum-based antiperspirant active is an astringent salt. The astringent salts include organic and inorganic salts of aluminum, zirconium, zinc, and mixtures thereof. The anion of the astringent salt can be, for example, sulfate, chloride, chlorohydroxide, alum, formate, lactate, benzyl sulfonate, or phenyl sulfonate. Exemplary classes of antiperspirant astringent salts include aluminum halides, aluminum hydroxyhalides, zirconyl oxyhalides, zirconyl hydroxyhalides, and mixtures thereof.

Exemplary aluminum salts include aluminum chloride and the aluminum hydroxyhalides having the general formula Al₂(OH)ₓQ_{y}.XH₂O, wherein Q is chlorine, bromine, or iodine; x is about 2 to about 5; x+y is about 6, wherein x and y are not necessarily integers; and X is about 1 to about 6. Exemplary zirconium compounds include zirconium oxy salts and zirconium hydroxy salts also referred to as zirconyl salts and zirconyl hydroxy salts, and represented by the general empirical formula ZrO(OH)_{2-nz}L_{z}, wherein z varies from about 0.9 to about 2 and is not necessarily an integer; n is the valence of L; 2-nz is greater than or equal to 0; and L is selected from the group consisting of halides, nitrate, sulfamate, sulfate, and mixtures thereof.

Exemplary aluminum-based antiperspirant actives, therefore, include, but are not limited to, aluminum bromohydrate, potassium alum, sodium aluminum chlorohydroxy lactate, aluminum sulfate, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octachloro-hydrex glycine complex, aluminum zirconium penta-chlorohydrex glycine complex, aluminum zirconium tetrachlorohydrex glycine complex, aluminum zirconium trichlorohydrex glycine complex, aluminum chlorohydrex PG, zirconium chlorohydrate, aluminum dichlorohydrate, aluminum dichlorohydrex PEG, aluminum dichlorohydrex PG, aluminum sesquichloro-hydrex PG, aluminum chloride, aluminum zirconium penta-chlorohydrate, chlorophyllin copper complex, numerous other useful antiperspirant compounds known in the art, and mixtures thereof.

In embodiments wherein an aluminum-based antiperspirant active is present in the composition, the amount of the aluminum-based antiperspirant active is decreased to achieve the same degree of odor control and sweat reduction as a composition that contains a larger amount of the aluminum-based antiperspirant active, but is free of polymeric microparticles.

A present composition also can contain a deodorant. A deodorant is a compound that reduces or masks unpleasant odors and helps protect against the formation of malodors on skin surfaces. Perfumes and the like can be used to mask the perception of malodor by the process of re-odorization. Absorbents can act as deodorants because of an ability to absorb malodorous chemicals. Examples of absorbent deodorants include, but are not limited to, bentonite, corn starch, diatomaceous earth, Fullers earth, hectorite, hydrated silica, kaolin, magnesium aluminum silicate, montmorillonite, potato starch, oat flour, rice starch, silica, talc, wheat starch, and mixtures thereof.

Exemplary odor control actives that can be incorporated into the composition include, but are not limited to, antimicrobial agents, antioxidants, enzyme inhibitors, odor absorbers and fragrances. Antimicrobial agents are a important category of the actives being used in the deodorant formulations, examples including but not limited to Triclosan, Tricocarban, Ethyhexyl glycerine, Decylene Glycol, Decalact Liquid (Sodium Caproyl/Lauroyl Lactyl Lactate Triethyl Citrate), Farnesol, SymDEO 125 (2-Methyl 5-Cyclohexylpentanol), cationic agents such as Benzalkonium chloride, Sensidin DO (mixture of Octenidine dihydrochloride, Ethylhexyl glycerine, Propylene Glycol), Tocopherol (vitamin E) and ascorbic acid (vitamin C) are widely used antioxidants to prevent oxidization of lipid in sweat to control odor formation. Zinc rincinoleate and resins are well known for their binding ability with odor causing fatty acid to control malodor. Different fragrances are used in deodorant products. Fragrance does not only provide a pleasant smell but also can mask malodor or control bacterial growth through carefully compounding of different fragrance components.

Cosmetic biocides also can be included in a present composition. Examples of cosmetic biocides include, but are not limited to, ammonium phenolsulfonate, benzalkonium chloride, benzethonium chloride, cetylpyridinium chloride, chlorophyllin-copper complex, chlorothymol, chloroxylenol, cloflucarban, dichloro-m-xylenol, methylbenzethonium chloride, phenol, sodium phenolsulfonate, triclocarban, triclosan, zinc phenolsulfonate, zinc ricinoleate, and mixtures thereof.

A composition of the present invention also can include 0%, or about 0.5%, to about 10%, and preferably about 1% to about 5%, by weight of the composition, of a surfactant, preferably a nonionic surfactant or a nonionic surfactant blend, having an HLB (hydrophilic lipophilic balance) value of about 3 to about 20. A surfactant can be included in the composition as a formulation aid, as a solvent, or for esthetics, for example. For emulsified compositions, the compositions contain a sufficient amount of surfactants of the proper identity and HLB value to provide a stable o/w or w/o emulsion. The selection of a surfactant to provide a stable emulsion is well within the capability of persons skilled in the art.

The HLB value of a particular nonionic surfactant can be found in McCutcheon's Emulsifiers and Detergents, North American and International Editions, MC Publishing Co., Glen Rock, N.J. (1993) (hereinafter McCutcheon's). Alternatively, the HLB value of a particular nonionic surfactant can be estimated by dividing the weight percent of oxyethylene in the surfactant by five (for surfactants including only ethoxy moieties). In addition, the HLB value of a nonionic surfactant blend can be determined by the following formula:

HLB=(wt. % A)(HLB_{A})+(wt. % B)(HLB_{B})ₙ

wherein wt. % A and wt. % B are the weight percent of nonionic surfactants A and B in the nonionic surfactant blend, and HLB_{A} and HLB_{B} are the HLB values for nonionic surfactants A and B, respectively.

Exemplary classes of nonionic surfactants include, but are not limited to, polyoxyethylene ethers of fatty (C₆-C₂₂) alcohols, polyoxypropylene ethers of fatty (C₆-C₂₂) alcohols, dimethicone copolyols, ethoxylated alkylphenols, polyethylene glycol ethers of methyl glucose, polyethylene glycol ethers of sorbitol, and mixtures thereof.

Exemplary nonionic surfactants having an HLB value of 10 or greater than can be used alone or in a nonionic surfactant blend include, but are not limited to, methyl gluceth-20, methyl gluceth-10, PEG-20 methyl glucose distearate, PEG-20 methyl glucose sesquistearate, PEG-200 castor oil, C₁₁₋₁₅pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-21 stearyl ether, polyoxyethylene-10 oleyl ether, polyoxyethylene-20 oleyl ether, an ethoxylated nonylphenol, ethoxylated octylphenol, ethoxylated dodecylphenol or ethoxylated fatty (C₆-C₂₂) alcohol including at least 9 ethylene oxide moieties, polyoxyethylene-20 isohexadecyl ether, dimethicone copolyol, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxyethylene-6 tridecyl ether, and mixtures thereof.

Exemplary nonionic surfactants having an HLB value of less than 10 that can be used alone or in a nonionic surfactant blend, include, but are not limited to, laureth-2, laureth-3, laureth-4, PEG-3 castor oil, an ethoxylated nonylphenol, ethoxylated octylphenol, ethoxylated dodecyphenol or ethoxylated fatty (C₆-C₂₂) alcohol having less than 9 ethylene oxide moieties, PEG-600 dioleate, PEG-400 dioleate, and mixtures thereof.

Numerous other nonionic surfactants having an HLB value of either about 10 or greater, or less than about 10, are disclosed in McCutcheon's at pages 1-246 and 266-272; in the CTFA International Cosmetic Ingredient Dictionary, Fourth Ed., Cosmetic, Toiletry and Fragrance Association, Washington, D.C. (1991) (hereinafter the CTFA Dictionary) at pages 1-651; and in the CTFA Handbook, at pages 86-94, each incorporated herein by reference.

In addition to nonionic surfactants, anionic or cationic surfactants can be used as the surfactant. Exemplary anionic surfactants, such as salts of fatty (C₈-C₂₂) acids, are disclosed in McCutcheon's at pages 263-266, incorporated herein by reference. Exemplary cationic surfactants are disclosed in McCutcheon's at pages 272-273 and in U.S. Patent Publication 2003/0086896, each incorporated herein by reference.

A present composition can contain a thickening, gelling, or suspending agent. In particular, the following compounds, both organic and inorganic, act primarily by thickening or gelling an aqueous portion of a composition:

acacia, agar, algin, alginic acid, ammonium alginate, ammonium chloride, ammonium sulfate, amylopectin, attapulgite, bentonite, C₉₋₁₅ alcohols, calcium acetate, calcium alginate, calcium carrageenan, calcium chloride, caprylic alcohol, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystallinc cellulose, montmorillonite, myristyl alcohol, oat flour, oleyl alcohol, palm kernel alcohol, pectin, PEG-2M, PEG-5M, polyvinyl alcohol, potassium alginate, potassium carrageenan, potassium chloride, potassium sulfate, potato starch, propylene glycol alginate, sodium carboxymethyl dextran, sodium carrageenan, sodium cellulose sulfate, sodium chloride, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, and mixtures thereof.

The following additional nonlimiting examples of gelling agents act primarily by thickening a nonaqueous portion of a composition:

abietyl alcohol, acrylinoleic acid, aluminum behenate, aluminum caprylate, aluminum dilinoleate, aluminum distearate, aluminum isostearates/laurates/palmitates or stearates, aluminum isostearates/myristates, aluminum isostearates/palmitates, aluminum isostearates/stearates, aluminum lanolate, aluminum myristates/palmitates, aluminum stearate, aluminum stearates, aluminum tristearate, beeswax, behenamide, behenyl alcohol, butadiene/acrylonitrile copolymer, a C₂₉₋₇₀ acid, calcium behenate, calcium stearate, candelilla wax, carnauba, ceresin, cholesterol, cholesteryl hydroxystearate, coconut alcohol, copal, diglyceryl stearate malate, dihydroabietyl alcohol, dimethyl lauramine oleate, dodecanedioic acid/cetearyl alcohol/glycol copolymer, erucamide, ethylcellulose, glyceryl triacetyl hydroxystearate, glyceryl triacetyl ricinoleate, glycol dibehenate, glycol dioctanoate, glycol distearate, hexanediol distearate, hydrogenated C₆₋₁₄ olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable glycerides, hydrogenated vegetable oil, hydroxypropylcellulose, isobutylene/isoprene copolymer, isocetyl stearoyl stearate, Japan wax, jojoba wax, lanolin alcohol, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhydride copolymer, octyldodecyl stearoyl stearate, oleamide, oleostearine, ouricury wax, oxidized polyethylene, ozokerite, palm kernel alcohol, paraffin, pentaerythrityl hydrogenated rosinate, pentaerythrityl rosinate, pentaerythrityl tetraabietate, pentaerythrityl tetrabehenate, pentaerythrityl tetraoctanoate, pentaerythrityl tetraoleate, pentaerythrityl tetrastearate, phthalic anhydride/glycerin/glycidyl decanoate copolymer, phthalic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eicosene copolymer, PVP/hexadecene copolymer, rice bran wax, stearalkonium bentonite, stearalkonium hectorite, stearamide, stearamide DEA-distearate, stearamide DIBA-stearate, stearamide MEA-stearate, stearone, stearyl alcohol, stearyl erucamide, stearyl stearate, stearyl stearoyl stearate, synthetic beeswax, synthetic wax, trihydroxystearin, triisononanoin, triisostearin, triisostearyl trilinoleate, trilaurin, trilinoleic acid, trilinolein, trimyristin, triolein, tripalmitin, tristearin, zinc laurate, zinc myristate, zinc neodecanoate, zinc rosinate, zinc stearate, and mixtures thereof.

In some embodiments, the thickening or gelling agents is, for example, acrylic and/or methacrylic polymers or copolymers, vinylcarboxylic polymers, polyglyceryl acrylates or methacrylates, polyacrylamides derivatives, cellulose or starch derivatives, chitin derivatives, alginates, hyaluronic acid and its salts, chonodroitin sulphates, xanthan, gellan, Rhamsan, karaya or guar gum, carob flour, and colloidal aluminum magnesium silicates of the montmorillonite type.

In other embodiments, the thickening or gelling agents include vinylcarboxylic polymers sold under the tradename CARBOPOL®, acrylic acid/ethyl acrylate copolymers, acrylic acid/stearyl methacrylate copolymers, carboxymethylcellulose, ethylcellulose, hydroxyethylcellulose, hydroxymethylcellulose, hydroxypropylcellulose, microcrystalline cellulose, hydroxypropyl guar, colloidal hectorites, bentonites, and the like.

A present composition also can contain a film-forming polymer. A film-forming polymer dries completely at room conditions to form a continuous film that helps deposit formulation components on the skin. Film forming polymers add conditioning and moisturizing benefits to the skin. An extensive list of film-forming polymers is disclosed in U.S. Patent Publication 2003/0049290, incorporated herein by reference in its entirety.

Additional components that can be formulated into a present composition, independently of loaded or unloaded polymeric microparticles, include, but are not limited to, one or more of (a) skin protectants to prevent or reduce chafing, skin irritation, and/or skin friction including, but not limited to, cod liver oil, cocoa butter, shark liver oil, and jojoba oil; (b) emollients to help maintain a soft, smooth, and pliable appearance of the skin, including, but not limited to, triglycerides such as vegetable oils (e.g., avocado oil, sunflower seed oil), esters such as oleyl oleate, isopropyl palmitate, and isopropyl myristate, lanolin and its derivatives, and phospholipids; (c) antimicrobial agents, including but not limited to, methyl paraben, sodium hydroxymethylglycinate, cyclic organic nitrogen compounds including imidazolidinedione compounds and poly-methoxybicyclic oxazolidone, phenyl and phenoxy compounds including benzyl alcohol, and y-phenoxyethanol, low molecular weight aldehydes including formaldehyde and glutaraldehyde, halogenated compounds including chlorhexidine, chlorobutanol, and dibromopropamidine, and mixtures thereof; (d) skin treatment agents to help repair and replenish the natural moisture barrier function of the epidermis including, but not limited to, niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, urea, gelatin, soluble collagen, royal jelly, tocopheryl nicotinate, and vitamin D3 and analogues or derivatives, and mixtures thereof; (e) plant extracts having an astringent effect for reducing the pore size including, but not limited to, clove extract, coix extract, witch hazel extract, and mixtures thereof; (f) anti-inflammatory agents including, but not limited to, a steroidal anti-inflammatory agent, such as hydrocortisone, and a non-steroidal anti-inflammatory agent; and (g) additional components such as colorants, cosmetic biocides, dyes and pigments, drying agents, denaturants, drug astringents, external analgesics, essential oils, fragrances, perfumes, soothing agents, skin healing agents, vitamins such as vitamin B6, and other natural extracts.

Opacifying agents, pearlescent agents, or fillers (e.g., titanium dioxide or a styrene/acrylamide latex) that render the composition nontransparent also can be included in the composition. The presence of such ingredients does not adversely affect the efficacy of the composition and are added to achieve a desired esthetic effect.

In addition to the essential ingredients and the optional ingredients described above, the present compositions also can include other optional ingredients traditionally included in antiperspirant compositions. These optional ingredients include, but are not limited to, dyes, fragrances, preservatives, antioxidants, detackifying agents, and similar types of compounds. These optional ingredients are included in the composition, individually, in a sufficient amount to perform its intended function, without adversely affecting the efficacy of the composition with respect to controlling reducing perspiration, and typically in an amount of about 0.01% to about 10%, by weight of the composition.

A present composition can be in the form of a solid, gel, lotion, or liquid for application to the skin by rubbing or spraying, for example. As such, a present composition can be a solution, dispersion, or emulsion. In another embodiment, a present composition can be applied to the skin from a wet wipe. Compositions of the present invention are topically applied directly to the skin. The composition is delivered by placing the composition into a dispenser and applying an effective amount by spraying or rubbing the composition onto the desired skin surface. A composition of the present invention also can be applied by using a pre-formed applicator, such as a roller, pad, sponge, tissue, or cotton ball.

In one embodiment, a wet wipe comprises a present composition that impregnates, coats, or is otherwise in contact with an insoluble substrate, for example, a woven or nonwoven fiber, fiber mixture, or foam of sufficient strength and absorbency to hold an effective amount of the deodorant composition.

Nonlimiting examples of suitable insoluble substrates include nonwoven substrates, woven substrates, hydroentangled substrates, air entangled substrates, natural sponges, synthetic sponges, polymeric netted meshes, and similar materials. Numerous non-limiting examples of insoluble substrates are disclosed in U.S. Patent Publication No. 2003/0049290, incorporated herein by reference in its entirety.

The amount of a present composition in a wet wipe is at least an amount sufficient to result in deposition of the composition onto the skin during application. The substrate therefore comprises the composition in an amount of about 100% to about 400%, more preferably about 150% to about 350%, still more preferably about 200% to about 300%, by weight of the substrate.

The following are non-limiting examples of embodiments of a composition of the present invention. As demonstrated in the examples, a present composition can be aqueous based, non-aqueous based, or an emulsion, either o/w or w/o. A composition of the present invention is prepared by techniques commonly known in the art. A person skilled in the art readily understands how to prepare a homogeneous or emulsified composition based on the formulations shown below.

All ingredients herein are based upon the total weight of the composition, and all such weight percentages are based on the active level and, therefore, do not include carriers that may be included in commercially available materials.

### EXAMPLE 1

| INCI Name | Wt. % |
|---|---|
| Cyclopentasiloxane | 35.4 |
| Dimethicone | 24.9 |
| Cyclohexasiloxane | 23.6 |
| Methyl Methacrylate/Glycol | 8.0 |
| Dimethacrylate Crosspolymer | |
| C18-36 Acid Triglyceride | 4.0 |
| Allyl Methacrylates Crosspolymer | 3.0 |
| Dimethicone/Vinyl dimethicone | 1.1 |
| Crosspolymer | |

### EXAMPLE 2

| INCI Name | Wt. % |
|---|---|
| Dimethicone | 25.00 |
| Cyclopentasiloxane | 24.60 |
| Methyl Methacrylate/Glycol | 18.00 |
| Dimethacrylate Crosspolymer | |
| Cyclohexasiloxane | 16.40 |
| Stearyl Alcohol | 8.00 |
| Petrolatum | 6.00 |
| Hydrogenated Castor Oil | 1.00 |
| Fragrance | 0.50 |
| Phenoxyethanol | 0.45 |
| Ethylhexylglycerin | 0.05 |

### EXAMPLE 3

### Comparative

| INCI Name | Wt. % |
|---|---|
| Cyclopentasiloxane | 38.05 |
| Dimethicone | 30.36 |
| Cyclohexasiloxane | 25.37 |
| C18-36 Acid Triglyceride | 4.88 |
| Dimethicone/Vinyl dimethicone | 1.34 |
| Crosspolymer | |

Example 1, Example 2, and Example 3 (Comparative) are non-aqueous compositions. Comparative Example 3 is free of polymeric microparticles.

The compositions of Example 1, Example 2, and Comparative Example 3 were evaluated for efficacy using the procedure defined by the Food and Drug Administration (FDA) Guidelines for Effectiveness Testing of OTC Antiperspirant in accordance with §350.60 of the Final Monograph entitled “Antiperspirant Drug Products For Over-the-Counter Human Use”, Final Rule, 21 CFR Parts 310, 350, and 369 (Federal Register/Vol. 64, No. 110/Monday, Jun. 9, 2003). In order to claim that a deodorant composition exhibits an “extra strength efficacy”, the composition must exhibit at least 30% sweat reduction in 50% of the panelists in the efficacy test performed according to the FDA monograph. An “efficacy” claim requires at least 20% sweat production in 50% of the panelists 24 hours after application, according to the FDA monograph.

Five individuals were recruited for the study after having refrained from using any antiperspirant products for at least 17 days. The individuals were instructed to wash their axilla before test product application. A test composition was applied to one axilla, while the contrallaterial axilla was used as an untreated control. The test composition was applied uniformly to cover a 4 cm (centimeter)×6 cm area centered in the axillary vault. The test composition was applied in an amount that a typical individual applies under normal use condition. No axillary antiperspirant was used by any individual during the treatment period, except for the text composition applied for the study. The effect of treatments on axillary sweating was evaluated by sweat collections made at one hour and 24 hours after application. Mean and median values were calculated to measure the central tendency of the adjusted perspiration ratio (Z) values.

The efficacy results are summarized in Table 1. The results clearly show that a present deodorant composition containing polymeric microparticles, as illustrated in Example 1, achieves more than 30% sweat reduction, which ranks the composition as “extra effective” based on the FDA guideline. A second composition containing polymeric microparticles, i.e., Example 2, imparts more than 20% sweat reduction, which ranks the composition as “effective” based on the FDA guidelines. The composition of Comparative Example 3, containing no polymeric microparticles, provides only a 13-15% sweat reduction, which is insufficient to rank the composition, or make a claim of, “effective for sweat reduction” based on the FDA guidelines.

TABLE 1
Five Subject Antiperspirant Efficacy

| | | | Comparative |
|---|---|---|---|
| Mean Sweat reduction % | Example 1 | Example 2 | Example 3 |
| 1 hour | 34.18 | n/a | 15.03 |
| 24 hours | 32.73 | 26.52 | 13.62 |

The following is a further example of a non-aqueous composition of the present invention.

### EXAMPLE 4

| INCL | Wt. % |
|---|---|
| Cyclopentasiloxane | 38.4 |
| Cyclohexasiloxane | 25.6 |
| Dimethicone | 24.9 |
| Allyl Methacrylates Crosspolymer | 6.0 |
| C18-36 Acid Triglyceride | 4.0 |
| Dimethicone/Vinyl dimethicone | 1.1 |
| Crosspolymer | |

The following is yet an additional example of a composition of the present invention. The aqueous composition of Example 5 imparts more than 20% sweat reduction when the polymeric microparticles are included. The classification of this composition is “effective” based on the FDA guidelines, as illustrated in Table 2.

### EXAMPLE 5

| INCI | Wt. % |
|---|---|
| Water | 87.05 |
| Methyl Methacrylate/Glycol | 8.00 |
| Dimethacrylate Crosspolymer | |
| Allyl Methacrylates Crosspolymer | 3.00 |
| Phenoxyethanol | 0.90 |
| Xanthan gum | 0.70 |
| Chlorphenesin | 0.25 |
| Ethylhexylglycerine | 0.10 |

TABLE 2
Five Subject Antiperspirant Efficacy

| Mean Sweat reduction % | Example 5 |
|---|---|
| 1 hour | 24.12 |
| 24 hours | 21.61 |

The following Examples 6 and 7 are emulsified o/w compositions of the present invention.

### EXAMPLE 6

### Emulsion

| INCI | Wt. % |
|---|---|
| Water | 64.8 |
| C12-15 Alkyl Benzoate | 8.0 |
| Methyl Methacrylate/Glycol | 8.0 |
| Dimethacrylate Crosspolymer | |
| Glycerine | 3.0 |
| Allyl Methacrylates Crosspolymer | 3.0 |
| PEG-20 Methyl Glucose Sesquistearate | 2.8 |
| Dimethicone | 2.0 |
| Butylene Glycol | 2.0 |
| Neopentyl Glycol Diethylhexanoate | 1.5 |
| Cetearyl Alcohol | 1.4 |
| Methyl Gluceth-20 | 1.0 |
| Phenoxyethanol | 0.9 |
| Methyl Glucose Sesquistearate | 0.7 |
| Ceteareth-20 | 0.6 |
| Xanthan gum | 0.2 |
| Ethylhexylglycerin | 0.1 |

### EXAMPLE 7

| INCI | Wt. % |
|---|---|
| Water | 68.30 |
| Methyl Methacrylate/Glycol | 8.00 |
| Dimethacrylate Crosspolymer | |
| C12-15 Alkyl Benzoate | 6.00 |
| Butylene Glycol | 5.00 |
| Glycerine | 3.00 |
| Dimethicone | 1.50 |
| Neopentyl Glycol Diethylhexanoate | 1.13 |
| PEG-100 Stearate | 1.05 |
| Glyceryl Stearate | 1.05 |
| Methyl Gluceth-20 | 1.00 |
| Phenoxylethanol | 0.90 |
| Stearyl Alcohol | 0.68 |
| Methyl Glucose Sesquistearate | 0.53 |
| Ethylhexylglycerine | 0.50 |
| Ceteareth-20 | 0.45 |
| Cetyl Alcohol | 0.38 |
| Chlorphenesin | 0.25 |
| Xanthan gum | 0.20 |
| Disodium EDTA | 0.10 |

The composition of Example 7 was evaluated for its effectiveness in reduced sweat production with 20 subjects using the procedure defined by FDA Guidelines for Effectiveness Testing of OTC Antiperspirant in accordance with §350.60 of the Final Monograph entitled “Antiperspirant Drug Products For Over-the-Counter Human Use”, Final Rule, 21 CFR Parts 310, 350, and 369 (Federal Register/Vol. 64, No. 110/Monday, Jun. 9, 2003). The composition, shown in Example 7, imparts more than 20% sweat reduction. The classification of this composition is “effective” based on the FDA guidelines, as illustrated in

TABLE 3
20 Subject Antiperspirant Efficacy

| Mean Sweat reduction % | Example 7 |
|---|---|
| 1 hour | 27.62 |
| 24 hours | 25.08 |

The above examples demonstrate a feature of embodiments of the disclosure, wherein the composition can be completely aqueous-based, aqueous and water-soluble solvent-based, hydrophobic solvent-based, or an emulsion, either o/w or w/o, and preferably o/w. Various conventional antiperspirant formulations avoided an aqueous carrier because the aqueous carrier adds to the feel of wetness and absorbents typically used in prior antiperspirant compositions tend to expand and form gels in the presence of water, making them unsuitable or difficult to formulate into an aqueous composition and retain antiperspirant properties. Compounds in accordance with embodiments of the present disclosure provide reduced wetness having an “extra effective” or “effective” ranking for sweat reduction efficacy, both one hour and 24 hours after application, even in the absence of an the aluminum-based antiperspirant active, such as an aluminum or zirconium salt. The improved sweat reduction may be attributed to the polymeric microparticles present in the composition. Without intending to be bound by theory, it is believed that the microparticles contained in a present composition reduce the generation of perspiration, in part, by adsorbing a portion of the perspiration. During an antiperspirant test, about 420 mg of test material was applied onto 4×6 cm2 underarm area. The composition of Example 7 contains 8% microparticles, thus approximately 33.6 mg of microparticles was applied to the test area. The applied microparticles would adsorb about 67 mg of oil and 67 mg of water. During an antiperspirant test, the test subjects need to produce at least 100 mg of perspiration in 20 mins per axilla and at least 600 mg difference of perspiration should be seen between the lowest and highest sweated subjects in 20 mins per axilla. Therefore over a 24 hour period of time, even the panelists that sweat the least would produce about 7200 mg of sweat. The capacity of the microparticles would only be 2% sweat produced and therefore would not be able to meet the 20% sweat reduction claim, even for panelists the produce the minimum sweat. Thus, a more than 20% sweat reduction observed in the antiperspirant test is beyond the adsorption capacity of microparticles. Without intending to be bound by theory, it is believed that the high surface area of microparticles may help facilitate and/or expedite the evaporation of perspiration and aid in the resulting reduction of perspiration beyond the adsorption capacity of the microparticles themselves.

Different amount of microparticles can be formulated into the present composition, the following examples show o/w emulsions comprising different amount of microparticles.

### EXAMPLE 8

| | A | B | C |
|---|---|---|---|
| INCI | Wt. % | Wt. % | Wt. % |
| DI water | 71.55 | 69.55 | 67.55 |
| C12-15 Alkyl Benzoate | 6.00 | 6.00 | 6.00 |
| Butylene Glycol | 5.00 | 5.00 | 5.00 |
| Methyl Methacrylate/Glycol | 4.00 | 6.00 | 8.00 |
| Dimethacrylate Crosspolymer | | | |
| Glycerine | 3.00 | 3.00 | 3.00 |
| Dimethicone | 1.50 | 1.50 | 1.50 |
| Neopentyl Glycol Diethylhexanoate | 1.13 | 1.13 | 1.13 |
| PEG-100 Stearate | 1.05 | 1.05 | 1.05 |
| Glyceryl Stearate | 1.05 | 1.05 | 1.05 |
| Methyl Gluceth-20 | 1.00 | 1.00 | 1.00 |
| Ceteareth-20 | 1.00 | 1.00 | 1.00 |
| Cetearyl alcohol | 1.00 | 1.00 | 1.00 |
| Phenoxylethanol | 0.90 | 0.90 | 0.90 |
| Methyl Glucose Sesquistearate | 0.53 | 0.53 | 0.53 |
| Ethylhexylglycerine | 0.50 | 0.50 | 0.50 |
| Chlorphenesin | 0.25 | 0.25 | 0.25 |
| Xanthan gum | 0.20 | 0.20 | 0.20 |
| Citric acid | 0.13 | 0.13 | 0.13 |
| Sodium Citrate | 0.11 | 0.11 | 0.11 |
| Disodium EDTA | 0.10 | 0.10 | 0.10 |

It is noted, however, that in some embodiments an aluminum-based antiperspirant active can be included in the composition, which can further improve sweat reduction. A composition in accordance with an embodiment of the disclosure and containing 4% Aluminum chlorohydrate is described in Example 8. The low amount of the aluminum-based antiperspirant active in the formula can beneficially reduce irritation from aluminum-based antiperspirant actives, without a reduction of the effectiveness of the composition for sweat reduction. In other embodiments, a silicon elastomer, like a dimethicone/vinyl dimethicone crosspolymer, is included in the composition to suspend the polymeric microparticles in the composition.

### EXAMPLE 9

| INCI | Wt. % |
|---|---|
| Water | 68.15 |
| Methyl Methacrylate/Glycol | 8.00 |
| Dimethacrylate Crosspolymer | |
| Butylene Glycol | 5.00 |
| PPG-15 Stearyl Ether | 4.00 |
| Aluminum Chlorohydrate | 4.00 |
| Glycerine | 3.00 |
| Steareth-2 | 2.50 |
| Steareth-21 | 1.00 |
| Cyclomethicone | 1.00 |
| Hydroxylpropyl Starch Phosphate | 1.00 |
| Fragrance | 1.00 |
| Phenoxyethanol | 0.90 |
| Chlorphenesin | 0.25 |
| Disodium EDTA | 0.10 |
| Ethylhexylglycerine | 0.10 |
| Sodium Hydroxide | q. s. |

A present deodorant composition, containing about 1% to about 20%, by weight, polymeric microparticles provides “effective” sweat reduction efficacy or “extra effective” sweat reduction efficacy to reduce wetness and control malodor formation. The present compositions can be free of an aluminum-based antiperspirant actives for example, aluminum chloride, aluminum chlorohydrate, aluminum zirconium tetrachlorohydrate gly, aluminum zirconium trichlorohydrate gly, aluminum sesquichlorodydrate, zirconyl hydroxychloride, aluminum zirconium pentachlorohydres gly, aluminum zirconium tetrachlorohydre gly and aluminum zirconium octochlorohydre gly, aluminum dichlorohydre PG, and aluminum dichlorohydre PG.

### EXAMPLE 10

| INCI | Wt. % |
|---|---|
| Water | 76.30 |
| C12-15 Alkyl Benzoate | 6.00 |
| Butylene Glycol | 5.00 |
| Glycerine | 3.00 |
| Dimethicone | 1.50 |
| Neopentyl Glycol Diethylhexanoate | 1.13 |
| PEG-100 Stearate | 1.05 |
| Glyceryl Stearate | 1.05 |
| Methyl Gluceth-20 | 1.00 |
| Phenoxylethanol | 0.90 |
| Stearyl Alcohol | 0.68 |
| Methyl Glucose Sesquistearate | 0.53 |
| Ethylhexylglycerine | 0.50 |
| Ceteareth-20 | 0.45 |
| Cetyl Alcohol | 0.38 |
| Chlorphenesin | 0.25 |
| Xanthan gum | 0.20 |
| Disodium EDTA | 0.10 |

The composition of Example 10 is the same product as described in Example 7 except the microparticle technology was removed from the product and was replaced by water. This effectively make this product a placebo to evaluate if the base formula causes sweat reduction when the microparticle technology is removed.

The composition of Example 7 was applied to panelists for four days in a row under the clinical conditions required by the FDA to prove antiperspirant efficacy. After the fourth day application of the product, the panelists were placed in a hot environment in order to simulate sweat and the reduction of sweat versus the baseline were evaluated one hour after the application of the product. The panelists than came back after 24 hours and the sweat reduction were evaluated again according the protocol described in the FDA monograph for antiperspirant activity.

### EXAMPLE 11

| Ingredient | Wt. % |
|---|---|
| DI water | 64.925 |
| Disodium EDTA | 0.100 |
| Methyl Gluceth-20 | 1.000 |
| Glycerine | 3.000 |
| Butylene Glycol | 5.000 |
| xanthan gum (CP keltrol 630) | 0.300 |
| Microsponge E101-07 | 8.000 |
| Chorophenesin | 0.250 |
| Lipomulse 165 | 2.100 |
| Methyl Glucose Sesquistearate, | 0.525 |
| Ceteareth-20, Emulgin B2 | 0.450 |
| Cetyl Alcohol | 0.375 |
| Stearyl Alcohol | 0.675 |
| Dimethicone 350 cst | 1.500 |
| Neopentyl Glycol Diethylhexanoate | 1.000 |
| 2-Methyl 5-Cyclohexylpentanol | 1.000 |
| Decylene Glycol | 0.300 |
| Decalact Liquid - Dr Straetmans | 1.000 |
| C12-15 Alkyl Benzoate | 6.000 |
| Euxyl 9010 | 1.000 |
| Flirt Fragrance 191740 | 1.500 |
| | 100.000 |

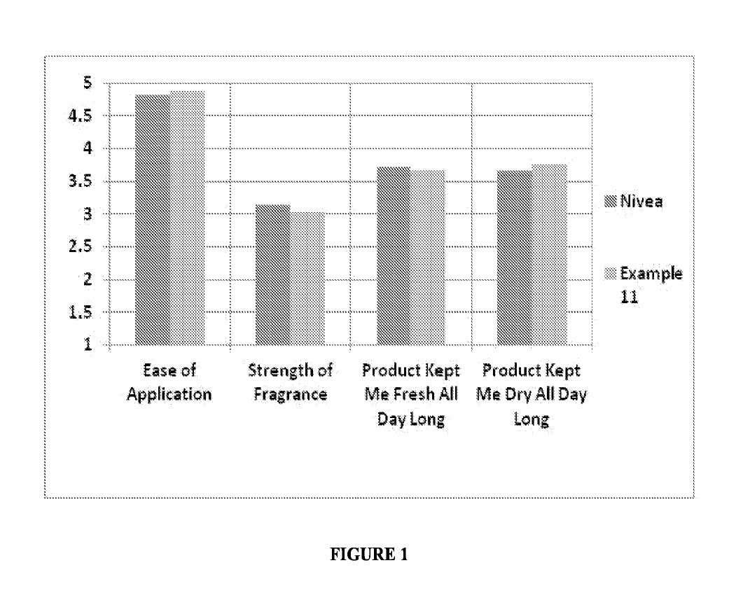

A consumer study was comparing the formula listed above to Nivea Product (Calm & Care) where both products were designed for use by women based on the fragrance of the product. The fragrance was developed by Firmenich to closely match that of the market product. The panelists were all chosen to be roll-on antiperspirant users. The panelists (53 completing the study) were first give the Nivea product to use for one week, and at the end of the week, they then switched to using the product of Example 11. At the end of the study, the panelist were asked to comment on the ease of application of the product, the strength of the fragrance, whether the product kept them fresh all day (odor control) and if product kept them dry all day long. The performance was rated on a scale between 1 and 5. For the ease of application question, the response was from 1 being difficult to use to 5 being easy to use. For strength of fragrance, the scale ranged from 1 (too weak), 3 (just right) to 5 (too strong). For the question of freshness and dryness, the scale was 1 (disagree), 3 (just right) and 5 (agree). The results are presented graphically in FIG. 1. A statistical comparison of the Nivea product to the product of this disclosure showed that the panelists considered products to be equivalent when using the T-test to evaluate the two sets of results.

As an additional benefit, because aluminum-based antiperspirant actives, such as aluminum or aluminum/zirconium salts, are not required in a present composition, the potential for skin irritation is greatly reduced. Effective sweat-reducing compositions free of an astringent salt provide benefits, such as reducing or eliminating contact dermatitis, and eliminating contact with aluminum salts that are connected with breast cancer, Alzheimer's disease, and clogged sweat pores leading to infected sweat glands. The polymeric microparticles also reduce greasiness of the composition to provide a dryer, powdery feel during use of application, thereby improving composition esthetics. In addition, by eliminating a source of perspiration-related malodors, the present compositions outperform deodorants that merely mask malodors.

Obviously, many modifications and variations of the invention as hereinbefore set forth can be made without departing from the spirit and scope thereof and, therefore, only such limitations should be imposed as are indicated by the appended claims.

1. A sweat reducing composition comprising:
an aluminum-based antiperspirant active in an amount of less than 15% by weight based on the total weight of the composition;
about 1% to about 20%, by weight, of water and oil adsorbing polymeric microparticles, and
a carrier.

2. A sweat reducing composition, consisting of:
water and oil adsorbing polymeric microparticles in an effective amount to reduce the generation of human perspiration in at least 50% of users tested by at least 10% one hour after contacting the skin and/or by at least 10% twenty-four hours after contacting the skin;
a carrier; and
optionally one or more of an aluminum-based antiperspirant active in amount less than 15% by weight, an odor control active material, an emollient, a fragrance, a dye, a moisturizer, a skin protectant, an antimicrobial, a cosmetic biocide, an opacifier, a surfactant, a gelling or thickening agent, a suspending agent, and a film-forming polymer,
wherein the reduction in perspiration is measured in accordance with the testing protocol defined at 21 CFR §350.60.

3. The composition of claim 2, wherein the microparticles are present in an effective amount to reduce the generation of human perspiration in at least 50% of users tested by at least 20% one hour after contacting the skin and/or by at least 20% twenty-four hours after contacting the skin.

4. The composition of claim 1, wherein the composition comprises the aluminum-based antiperspirant active in an amount of less than 10% by weight.

5. The composition of claim 1, further comprising an odor control active material.

6. The composition of claim 5, wherein the odor control active material is selected from the group consisting of antimicrobial agents, antioxidants, enzyme inhibitors, odor absorbers, fragrances, and combinations thereof.

7. The composition of claim 1, wherein the polymeric microparticles are highly crosslinked and are derived from methacrylate monomers, acrylate monomers, or mixtures thereof.

8. The composition of claim 1, wherein the polymeric microparticles comprise an allyl methacrylate crosspolymer, an ethylene glycol dimethacrylate/allyl methacrylate copolymer, a lauryl methacrylate/ethylene glycol dimethacrylate copolymer, a methyl methacrylate/glycol dimethacrylate copolymer, and mixtures thereof.

9. The composition of claim 1, wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

10. (canceled)

11. (canceled)

12. The composition of claim 1, wherein the polymeric microparticles are free of an added aluminum-based antiperspirant active.

13. The composition of claim 1, wherein the polymeric microparticles are loaded with a cosmetic or therapeutic compound.

14. The composition of claim 13, wherein the polymeric microparticles are loaded with about 10% to 90%, by weight of the loaded polymeric microparticles, of the cosmetic or therapeutic compound.

15. The composition of claim 13, wherein the polymeric microparticles are loaded with a cosmetic or therapeutic compound selected from the group consisting of skin protecting agent, moisturizing ingredients, odor control agents, fragrance, skin healing agents, and combinations thereof.

16. The composition of claim 1, wherein the carrier comprises water.

17. The composition of claim 16 wherein the carrier further comprises a water-soluble organic solvent.

18. The composition of claim 1, wherein the carrier comprises a volatile silicone, a volatile hydrocarbon, or a mixture thereof.

19. The composition of claim 18, wherein the carrier further comprises a nonvolatile silicone, a nonvolatile hydrocarbon, or a mixture thereof.

20. The composition of claim 1, wherein the composition is in a form of a water-in-oil emulsion or an oil-in-water emulsion.

21. The composition of claim 1 wherein the composition further comprises a suspending agent for the polymeric microparticles.

22. The composition of claim 21, wherein the suspending agent comprises a silicone elastomer, a gum or polymeric thickening agent.

23. The composition of claim 1 further comprising an aluminum-based antiperspirant active, a deodorant, or a mixture thereof, in an amount of less than 10%, by weight of the composition.

24. The composition of claim 23 wherein the antiperspirant active comprises aluminum chloride, aluminum chlorohydrate, aluminum-zirconium or a mixture thereof.

25. The composition of claim 24, wherein the aluminum-based antiperspirant active is selected from the group consisting of aluminum chloride, aluminum chlorohydrate, aluminum chlorohydrex, aluminum chlorohydrex propylene glycol, aluminum dichlorohydrate, aluminum dicholorohydraex polyethylene glycol, aluminum sesquichlorohydrate, aluminum sesquichlorohydrex polyethylene glycol, aluminum zirconium octachlorohydrex gly, aluminum zirconium pentachlorohydrate, aluminum zirconium pentachlorohydrex gly, aluminum zirconium tetrachlorohydrate, aluminum zirconium tetrachlorohydrex gly, aluminum zirconium trichlorohydrate, aluminum zirconium tricholorhydrex gly, and combinations thereof.

26. The composition of claim 1 further comprising one or more of an emollient, a fragrance, a dye, a moisturizer, a skin protectant, an antimicrobial, a cosmetic biocide, an opacifier, a surfactant, a gelling or thickening agent, a suspending agent, and a film-forming polymer.

27. The composition of claim 1, wherein the composition is in the form of a solid, a gel, or a liquid.

28. A wet wipe comprising:
a water-insoluble substrate; and
the composition of any claim 1.

29. The wet wipe of claim 28, wherein the composition is present in an amount of about 100% to about 400% by weight of the substrate.

30. A method of reducing the generation of human perspiration, comprising:
contacting human skin with a composition comprising:
less than 15% by weight of an aluminum-based antiperspirant active;
about 1% to about 20%, by weight, of water and oil adsorbing polymeric microparticles, and
a carrier,
wherein the composition is applied in an effective amount to reduce the generation of human perspiration.

31. A method of reducing the generation of human perspiration, comprising
contacting human skin with a composition comprising about 1% to about 20%, by weight, of water and oil adsorbing polymeric microparticles and a carrier, in an effective amount to reduce the generation of human perspiration,
wherein the composition provides a reduction in the perspiration that is greater than the adsorbent capacity of the polymeric microparticles.

32. The method of claim 31, wherein:
the composition provides an at least 20% reduction in the perspiration in 24 hours for at least 50% of the users tested, and
the microparticles adsorb less than 25% of the perspiration based on the amount perspiration reduced in order to provide at least 20% reduction in perspiration as measured in accordance with the testing protocol defined at 21 C.F.R. §350.60.

33. (canceled)

34. The method of claim 31, wherein the composition further comprises less than 15% by weight of an aluminum-based antiperspirant active.

35. The method claim 30, wherein the composition is substantially free of an aluminum-based antiperspirant active and provides a reduction in perspiration that is comparable to a composition comprising a therapeutically effective amount of an aluminum-based antiperspirant active to reduce human perspiration.

36. The method of claim 30, wherein the perspiration in at least 50% of users tested is reduced by at least 10% one hour after contacting the skin as measured in accordance with the testing protocol defined at 21 C.F.R. §350.60.

37. (canceled)

38. (canceled)

39. (canceled)

40. (canceled)

41. (canceled)
